(19) United States
(12) Patent Application Publication
(10) Pub. No.: US 2012/0143001 A1
(43) Pub. Date: Jun. 7, 2012

(54) Tasting Dildo

(76) Inventor: Jesse K. Case, Norwalk, CT (US)

(21) Appl. No.: 12/961,224

(22) Filed: Dec. 6, 2010

(51) Int. Cl. A61F 5/00; A61F 5/44; A61H 1/00

(52) U.S. Cl. 600/38; 601/46; 604/330

(57) ABSTRACT

A sexual aid or dildo that is used to collect fluid from a woman's vagina before, during or after a woman's orgasm. A method of using a sexual aid or dildo for tasting fluid from a woman's vagina for increased sexual pleasure. A method of using a dildo for same sex or male to male sexual pleasure.

### FIELD OF THE INVENTION

This invention relates generally to the field of sexual aids and more specifically to a dildo used to collect a woman's fluid from ejaculation created by an orgasm.

### BACKGROUND OF THE INVENTION

Devices for aiding in the pleasure of sexual experiences for both men and women have been in existence for many years. One such device is known as a dildo. This device is typically a penis shaped member than can be inserted into the vagina of a woman, or in some cases, into the anus of a man.

Many different shapes and sizes of dildos are available in the market today. Some existing dildos have the ability to vibrate and include a vibrating means to increase sexual pleasure and to provide additional sensation for a user.

Although dildos, including vibrating types, do provide pleasure for their users, there is a deficiency in that there has never been a way to collect fluid from a woman's ejaculation created by an orgasm via a dildo. There also has never been a way to collect a woman's ejaculation or vaginal fluid before or after orgasm by using a dildo. During sexual experiences, it may be desirable to collect a woman's ejaculation before, during, and/or after orgasm for tasting. Collecting a woman's ejaculation from orgasm may provide maximum oral pleasure during sexual experiences. It is thus, desirable to collect a woman's ejaculation from orgasm to increase sexual pleasure and this is an object of the present invention.

Despite the various apparatus in the prior art, there has heretofore not been provided a suitable and attractive solution to the problem of being able to collect a woman's fluid from before, during or after orgasm. There has also heretofore not been provided a suitable and attractive solution to the problem of being able to collect a woman's fluid during sexual experiences.

U.S. Pat. No. 6,945,927 (Ritchie et al.) discloses a dildo having a head with two lobes and an external groove/channel that extends from the head partly down or entirely down the length of the shaft of the dildo. This dildo is used for stimulating the upper vaginal wall of a vagina. However, Ritchie et al. do not disclose having reservoirs, specifically reservoirs located on the distal tip or base of the head portion, and is not used for collecting fluid or for tasting the fluid from a woman's ejaculation from orgasm.

Other prior art references, such as U.S. Pat. No. 6,896,653 (Vail, III et al.) disclose a personal pelvic viewer for a woman and U.S. Pat. No. 4,534,362 (Schumacher et al.) discloses an apparatus that teaches the collection of vaginal fluid to determine fertility. However, these prior art references do not provide for the disclosure of reservoirs located on the distal tip or base of the head portion of a dildo for the collection of a woman's fluid from before, during or after orgasm.

Therefore it is desirable to provide a dildo which obviates the foregoing and other limitations and disadvantages of prior art dildos, and to provide a dildo for tasting a woman's orgasm to increase the pleasure of sexual experiences.

### SUMMARY OF THE INVENTION

Thus, it is an object of the present invention to provide an improved dildo that allows a user to collect the fluid from a woman's orgasm.

Another object of the invention is to provide an improved dildo that may collect fluid from two women at a time or from more than one woman.

Other objects and advantages of the invention will become apparent from the following descriptions, taken in connection with the accompanying drawings, wherein, by way of illustration and example, an embodiment of the present invention is disclosed.

Objectives of the invention are achieved by providing a dildo comprising a longitudinal shaft and a head portion, the head portion located at the distal end of the longitudinal shaft, the head portion having one or more reservoirs for collecting fluid.

At least one of the one or more reservoirs may be located at the distal tip of the head portion. Furthermore, at least one of the one or more reservoirs may be located at the base of the head portion. The one or more reservoirs may be concave shaped or may be shaped as a cup for collection. The longitudinal shaft may be substantially cylindrical and the longitudinal shaft may be hollow. The head portion of the dildo may also be shaped similarly to the glans penis of the human male.

In other embodiments, the dildo may comprise one or more channels. The one or more channels may extend through the head portion of the dildo. At least one of the one or more channels may connect at least one of the one or more reservoirs to each other. The channels may be of various shapes and orientations, may be both internal and/or external to the head of the dildo. Furthermore, the channels may be oriented perpendicular to or parallel to the horizontal shaft, may be spiral, may be dimple shaped, and/or may be groove shaped.

Embodiments of various shapes and combination of channels may be used with each other. For example, the head of the dildo may have dimples and spiral channels, and may have horizontal channels and vertical grooves together. Other combinations are also possible.

In other embodiments, the one or more reservoirs have multiple depressions or indentations. The one or more reservoirs may be made of a sticky material. The head portion of the dildo may have dimples located on the surface of the head portion. Fluid may collect in the dimples improving the ability for tasting.

In other embodiments, the dildo may further comprise a second head portion, the second head portion located on the proximal end of the longitudinal shaft. The second head portion may have one or more reservoirs for collecting fluid. The reservoirs located on the second head portion may be located on the tip of the second head portion, or may be located on the base of the second head portion. The second head portion may have dimples, grooves, spiral channels, horizontal channels and may be made of a sticky material.

The dildo may be used to collect fluid form a female's vagina, before, during and after organism. In other embodiments, the dildo may be inserted into the anus of a male or female. The dildo may be used for other purposes, such as inserting the dildo into a female or male's mouth as well.

In other advantageous embodiments, the head portion may vibrate. The dildo may comprise a power supply and motor within the longitudinal shaft, the power supply and motor allowing the dildo to vibrate. The motor may be controlled by a combination switch and a potentiometer, the potentiometer located at the proximal end of the longitudinal shaft.

The dildo may further comprise electrical wiring that connects the potentiometer, the motor and the power supply, the electrical wiring being inside the longitudinal shaft.

In other embodiments, the head portion of the dildo may have ribbed indentations on the sides.

In certain advantageous embodiments, the longitudinal shaft and the head portion are fused together as one piece. In other advantageous embodiments, the longitudinal shaft and the head portion are formed as two separate pieces that can be screwed or attached to each other. This allows for easier cleaning of the pieces.

Other objectives of the invention are achieved by providing a dildo for collecting a female's fluid from ejaculation comprising: a longitudinal shaft and a head portion, the head portion located at a distal end of the longitudinal shaft, the head portion having one or more reservoirs for collecting fluid, wherein at least one of the one or more reservoirs is located at a distal tip of the head portion, and a second head portion, the second head portion located on the proximal end of the longitudinal shaft, the second head portion having one or more reservoirs for collecting fluid, wherein at least one of the one or more reservoirs is located at a distal tip of the second head portion.

Other objectives of the invention are achieved by providing a method for tasting fluid from a woman's vagina comprising: providing a dildo with a longitudinal shaft and a head portion, the head portion located at a distal end of the longitudinal shaft, the head portion having one or more reservoirs for collecting fluid; inserting the head portion into a female's vagina causing the female to orgasm; collecting fluid from the female's vagina; and allowing a user to taste the collected fluid.

The invention is not limited to these embodiments, as other combinations are contemplated by the invention. Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a perspective view of an embodiment of a dildo of the present invention;

FIG. 2 is a side view of the dildo of FIG. 1;

FIG. 3 is an end view from of the head of the dildo of FIG. 1;

FIG. 4 is an end view of base of the shaft of the dildo of FIG. 1;

FIG. 5 is a cross sectional view of the dildo of FIG. 1;

FIG. 6 is a perspective view of the head of the dildo of FIG. 1;

FIG. 7 is a double end dildo substantially as shown in FIG. 1, but incorporating two heads;

FIG. 8 is a perspective view of an embodiment of the head of the dildo of FIG. 1;

FIG. 9 is a perspective view of an embodiment of the head of the dildo of FIG. 1;

FIG. 10 is a perspective view of an embodiment of the head of the dildo of FIG. 1;

FIG. 11 is a perspective view of an embodiment of the head of the dildo of FIG. 1;

FIG. 12 is a perspective view of an embodiment of the head of the dildo of FIG. 1;

FIG. 13A is a perspective view of an embodiment of the head of the dildo of FIG. 1; and

FIG. 13B is a cross sectional view of the head of the dildo of FIG. 13A.

### DETAILED DESCRIPTION OF THE INVENTION

Advantageous embodiments of the invention are directed to a dildo or vibrator device that allows for a person to taste the fluids created by orgasm of a woman. This is done by having fluid be collected by the device, more specifically at the collection tip, so that it can be tasted.

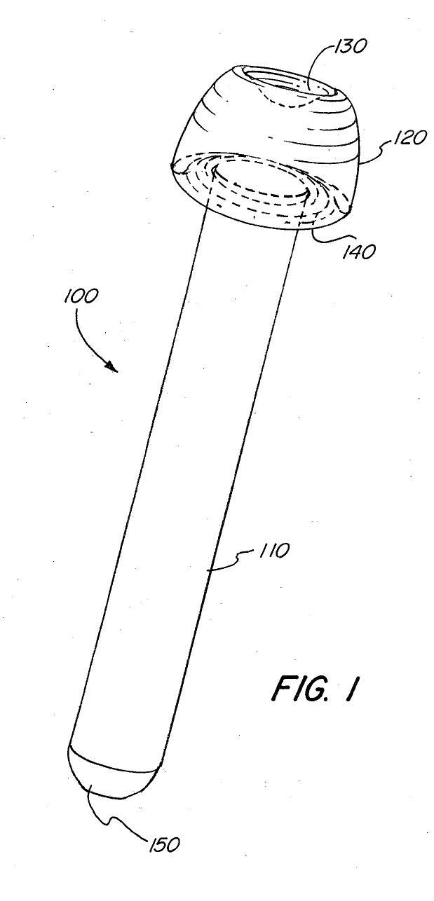

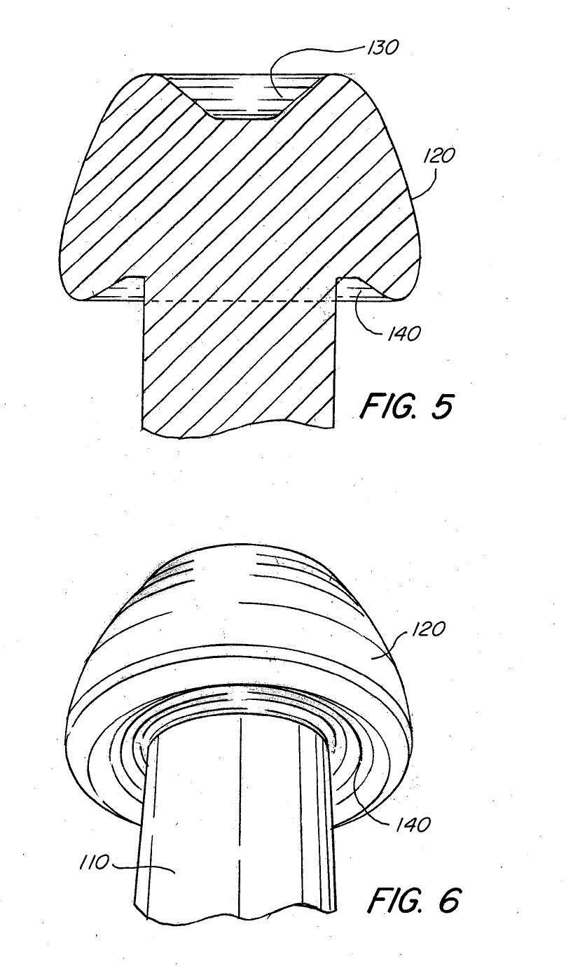

In embodiments of the invention, shown in the enclosed FIGS. 1-13, the dildo is designed as a concave tipped distal head. It has a collection tip 130 also known as a reservoir at the top of the dildo, as most clearly shown in FIG. 5. There is an additional collection tip/reservoir at the bottom of the dildo head as shown most clearly in FIG. 6 that allows fluid to be collected as the dildo is removed from a woman's vulva or vagina.

The reservoir described in this invention has a depth to it and is used to collect fluid. The reservoir or collection tip is more than just a surface indentation and is defined as being a cavity that allows fluid to collect within the reservoir.

The reservoir may have a certain ratio to the concave tipped distal head whereby the depth of the reservoir extends is one quarter of the total depth of the concave tipped distal head. In other embodiments, the depth of the reservoir may be up to one third the total depth of the concave tipped distal head. In other embodiments, the depth of the reservoir may be less than one quarter of the total depth of the concave tipped distal head.

In other embodiments, the reservoir may have sufficient depth to collect a suitable amount of female fluid for tasting.

Other embodiments of the invention involve different shapes for the collection tip or reservoir. The tip can be of a trough form or may have multiple troughs. The collection tip may be made of a sticky material that allows fluid to stick to the tip. The collection tip may have channels that run through it from the top of the collection tip to the bottom of the distal head.

Other embodiments may involve the distal head having holes so that fluid can be collected.

The dildo is typically made of a rubber material, although other material can be used. Jelly or other such petroleum products may be used with the dildo. The material used for the dildo is preferably sticky so that fluid can be collected. The dildo can be made of multiple rubbers and other such materials that are insulators and are smooth.

In other embodiments, the dildo may have multiple heads (such as a two-headed dildo). Functionally, the dildo may be able to vibrate, may be used during male-women intercourse, women-woman intercourse, or male-male intercourse. The device may also be used for orgies or sexual situations where more than two people are involved. The device may also be used for solo use when a female wants to taste her own fluids created by orgasm.

The vibration of the dildo may be powered by a battery. The battery may be located at the bottom of the dildo as well as inside its mid-section.

Referring to FIG. 1, dildo 100 is shown having a shaft 110, head portion 120, and end 150, the end 150 being removeable to allow for a battery or power source to be placed within shaft 110, allowing the dildo 100 to vibrate. The battery or power source is not shown.

The dildo has a reservoir 130 located at the distal tip of head portion 120. The dildo has a reservoir 140 located at the base of head portion 120. Both reservoirs 130 and 140 are used to collect a woman's vaginal fluid from before, during or after orgasm for tasting purposes.

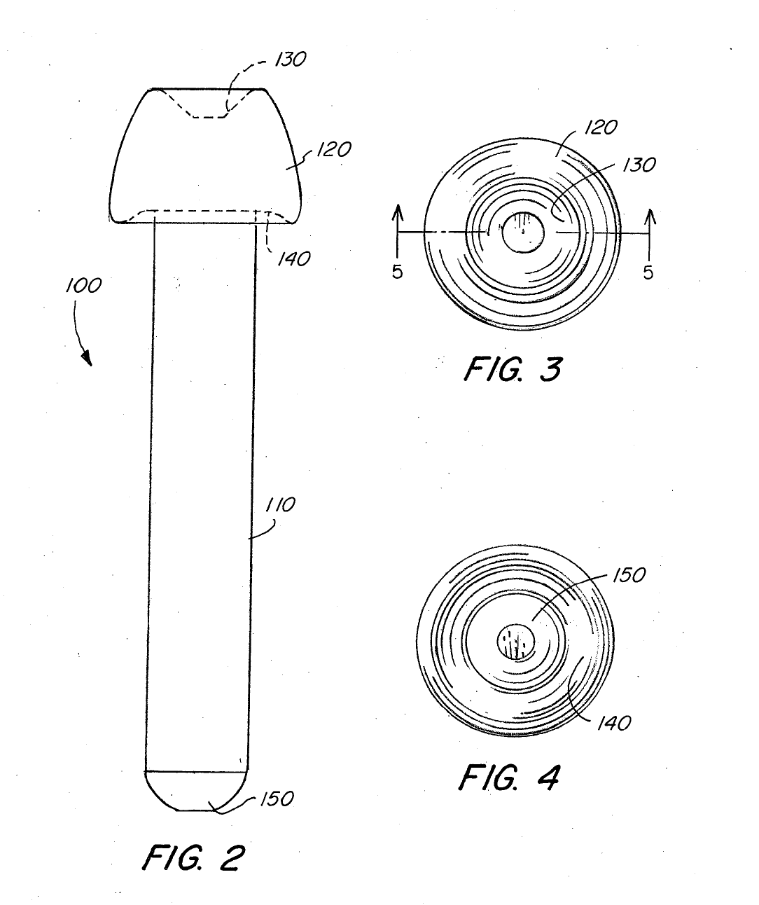

FIG. 2 is a side view of the dildo 100 of FIG. 1. In FIG. 2, the shaft 110, end 150, and head 120 having two reservoirs 130 and 140 are shown. The reservoirs 130 and 140 are shown as having a concave shape, however, various other reservoir shapes are possible. The reservoirs may have a trough shape, or may have a shape with multiple troughs for example.

FIG. 3 is an end view of the head portion 120, which corresponds to head 120 shown in FIGS. 1-2. Here, reservoir 130 is shown on head 120. FIG. 4 is an end view of the end of the shaft 110 whereby end 150 is shown and also reservoir 140, which is present at the base of the head of the dildo 120, which has a larger diameter than the shaft 110.

FIG. 5 and FIG. 6 show a cross sectional view of the dildo of FIG. 1 and a perspective view of the head of the dildo of FIG. 1, respectively.

In FIG. 5, head portion 120 is shown having a reservoir 130 located at the distal tip of head portion 120 and reservoir 140 shown located at the base of the distal tip of head portion 120. In other embodiments, the reservoir 130 may be replaced by various other reservoir shapes such as troughs and multiple troughs, for example, which may define the reservoir.

FIG. 6 shows the head portion 120 and shaft portion 110 and clearly shows the reservoir 140 located at the base of the distal tip of head portion 120. The reservoir 140 may have multiple shapes and is used for collecting fluid from a woman's vagina for tasting. This reservoir is shown having a diameter which tapers towards the intersection with the shaft. The head portion 120 is shown as being mushroom shaped.

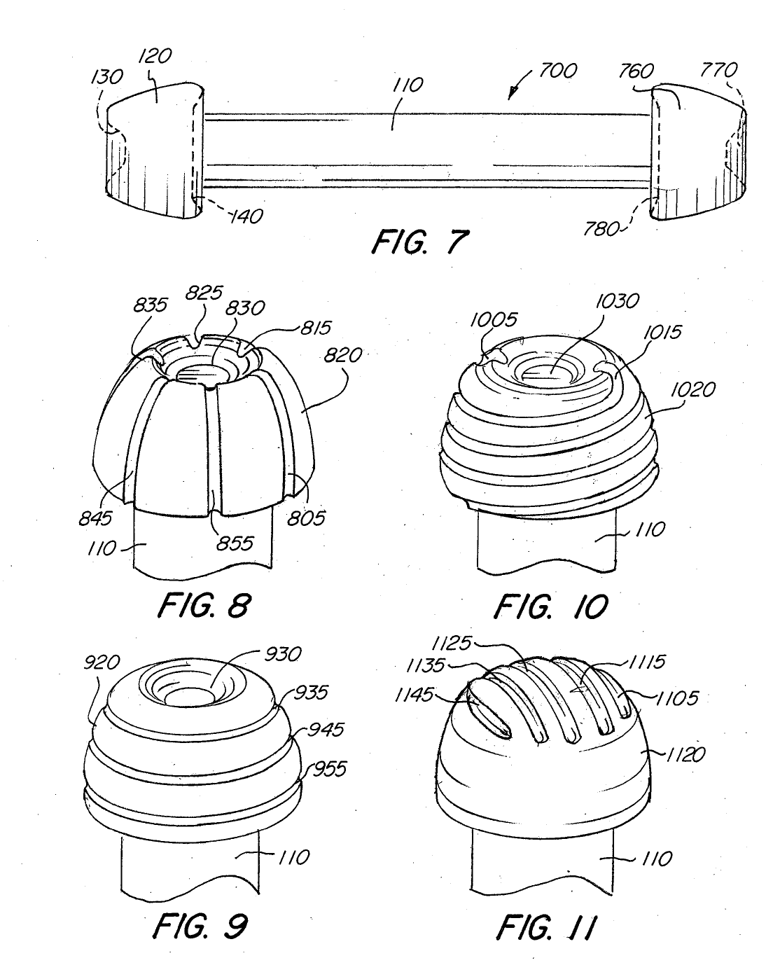

FIG. 7 shows a double sided dildo 700 having a shaft 110 and two head portions 120 and 760. Reservoir 130 is shown located at the distal tip of head portion 120 and reservoir 140 shown located at the base of the distal tip of head portion 120. Reservoir 770 is shown located at the distal tip of head portion 760 and reservoir 780 shown located at the base of the distal tip of head portion 760. This dildo 700 may be used with two females simultaneously or may be used with a female and a male together, or for either single use with a male or female.

FIGS. 8-13 show different embodiments of the head portions of various embodiments of the present invention.

FIG. 8 shows head portion 820 connected to shaft portion 110. Here, head portion 820 has reservoir 830. Also shown are channels 805, 815, 825, 835, and 845. In certain embodiments, these channels are used to connect the reservoir 830 to second reservoir. This second reservoir is similar to reservoir 140 provided in other figures. The channels also are used to increase the tasting capabilities of the dildo. In certain embodiments there are more than the six channels shown and other embodiments provide for less than six channels.

Other embodiments may involve having a dildo with only channels 805, 815, 825, 835, 845 and 855 for tasting.

FIG. 9 shows head portion 920 connected to shaft portion 110. Here, head portion 920 has reservoir 930. Also shown are channels 935, 945, and 955. These channels are shown as being approximately perpendicular to shaft 910 of the dildo. The channels are used to increase the tasting capabilities of the dildo. In certain embodiments there are more than the three channels shown and other embodiments provide for less than three channels. In certain embodiments, these channels are used to connect the reservoir 930 to second reservoir. This second reservoir is similar to reservoir 140 provided in other figures.

Other embodiments may involve having a dildo without any reservoirs, such that the dildos only have channels 935, 945 and 955 for tasting.

FIG. 10 shows head portion 1020 connected to shaft portion 110. Here, head portion 1020 has reservoir 1030. Also shown are channels 1005 and 1015, which are shown as spirals going from reservoir 1030 and spiraling down through the head portion 1020. The channels are used to increase the tasting capabilities of the dildo. This embodiment may or may not have second reservoir at the base of the head portion 1020. In certain embodiments there are more than the two spiraling channels shown and other embodiments provide for less than two spiraling channels.

Other embodiments may involve having a dildo without any reservoirs, such that the dildos only have channels 1005 and 115 for tasting.

FIG. 11 shows head portion 1120 connected to shaft portion 110. Here, channels 1105, 115, 1125, 1135 and 1145 are shown. The channels are used to increase the tasting capabilities of the dildo. This embodiment may or may not have reservoir at the base of the head portion 1120. In certain embodiments there are more than the five channels shown and other embodiments provide for less than five channels. This embodiment does not have a reservoir located at the distal tip of the head, but rather has channels in its place.

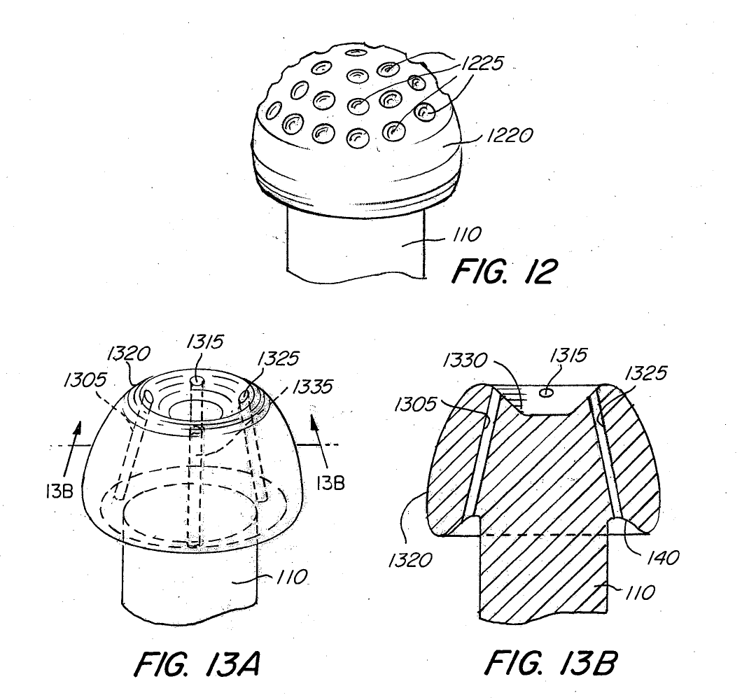

FIG. 12 shows head portion 1220 connected to shaft portion 110. Here dimples 1225 are shown. The dimples 1225 are used to increase the tasting capabilities of the dildo. This embodiment may or may not have reservoir at the base of the head portion 1220.

FIG. 13A shows head portion 1320 connected to shaft portion 110. Here, internal channels 1305, 1314, 1325 and 1335 are shown as well as reservoir 1330 at the distal tip of head portion 1320. These channels are internal channels and connect to reservoir 140.

FIG. 13B shows a cross section view of FIG. 13 where reservoir 1330 is shown with channels 1305 and 1325 connecting reservoir 1330 to reservoir 140. This provides for additional and improved tasting of vaginal fluid.

While the invention has been specifically described in connection with certain specific embodiments thereof, it is to be understood that this is by way of illustration and not of limitation and that various changes and modifications in form and details may be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

What is claimed is:

1. A dildo comprising:
a longitudinal shaft and a head portion, the head portion located at the distal end of the longitudinal shaft, the head portion having one or more reservoirs for collecting fluid.

2. The dildo of claim 1, wherein at least one of the one or more reservoirs is located at the distal tip of the head portion.

3. The dildo of claim 1, wherein at least one of the one or more reservoirs is located at the base of the head portion.

4. The dildo of claim 1, wherein the head portion is shaped similarly to the glans penis of the human male.

5. The dildo of claim 1, further comprising one or more channels, the one or more channels extending through the head portion of the dildo.

6. The dildo of claim 6, wherein at least one of the one or more channels connect at least one of the one or more reservoirs to each other.

7. The dildo of claim 1, wherein the one or more reservoirs have multiple depressions.

8. The dildo of claim 1, wherein the one or more reservoirs are made of a sticky material.

9. The dildo of claim 1, wherein the head portion has dimples located on the surface of the head portion.

10. The dildo of claim 1, further comprising a second head portion, the second head portion located on the proximal end of the longitudinal shaft.

11. The dildo of claim 10, wherein the second head portion has one or more reservoirs for collecting fluid.

12. The dildo of claim 1, wherein the fluid is from a female orgasm.

13. The dildo of claim 1, wherein the head portion vibrates.

14. The dildo of claim 1, further comprising a power supply and motor within the longitudinal shaft.

15. The dildo of claim 14, wherein the motor is controlled by a combination switch and a potentiometer, the potentiometer located at the proximal end of the longitudinal shaft.

16. The dildo of claim 15, further comprising electrical wiring that connects the potentiometer, the motor and the power supply, the electrical wiring being inside the longitudinal shaft.

17. The dildo of claim 1, wherein the head portion had ribbed indentations on a side of the head portion.

18. The dildo of claim 1, wherein the one or more reservoirs are concave shaped.

19. The dildo of claim 1, wherein the longitudinal shaft is substantially cylindrical.

20. The dildo of claim 1, wherein the longitudinal shaft and the head portion are fused together as one piece.

21. A dildo for collecting fluid from a female orgasm comprising:
a longitudinal shaft and a head portion, the head portion located at a distal end of the longitudinal shaft, the head portion having one or more reservoirs for collecting fluid, wherein at least one of the one or more reservoirs is located at a distal tip of the head portion, and
a second head portion, the second head portion located on a proximal end of the longitudinal shaft, the second head portion having one or more reservoirs for collecting fluid, wherein at least one of the one or more reservoirs is located at the distal tip of the second head portion.

22. A method for tasting fluid from a female's vagina comprising:
providing a dildo with a longitudinal shaft and a head portion, the head portion located at a distal end of the longitudinal shaft, the head portion having one or more reservoirs for collecting fluid;
inserting the head portion into a female's vagina causing the female to orgasm;
collecting fluid from the female's vagina; and
allowing a user to taste the collected fluid.

23. The method of claim 22, wherein the step of inserting the head portion into a female's vagina causing the female to orgasm is done repeatedly.
